(19) United States
(12) Patent Application Publication
(10) Pub. No.: US 2014/0271603 A1
(43) Pub. Date: Sep. 18, 2014

(54) METHOD FOR PREPARING HUMAN FACTOR H

(71) Applicant: LABORATOIRE FRANCAIS DU FRACTIONNEMENT ET DES BIOTECHNOLOGIES, Courtaboeuf Cedex (FR)

(72) Inventor: Toufik Abache, Santes (FR)

(21) Appl. No.: 14/353,595

(22) PCT Filed: Oct. 25, 2012
(86) PCT No.: PCT/FR2012/052461

§ 371 Date: Apr. 23, 2014

(30) Foreign Application Priority Data
Oct. 25, 2011 (FR) 11/59686

(51) Int. Cl. C12N 9/88

(52) U.S. Cl. CPC C12N 9/88
USPC 424/94.5; 536/23.2; 435/232; 435/320.1; 435/366

(57) ABSTRACT

A human cell line for implementing a method for preparing recombinant human factor H, more particularly the recombinant human factor H represented by the sequence SEQ ID NO: 1, or a variant possessing a percentage homology of at least 99% with the sequence SEQ ID NO: 1, with a yield greater than the quantity of endogenous factor H produced by said cell line.

The present invention relates to a method for preparing recombinant human factor H in a human cell line, with an improved yield.

Factor H is a plasma protein present in human plasma at an average level of 500 mg/L. It is mainly produced constitutively by the liver cells, but is also produced locally by other cells, such as the epithelial cells of the pigment of the retina, endothelial cells, platelets or mesenchymal stem cells.

The main function of factor H is regulation of the activation of the alternative pathway of complement. Factor H is involved in this regulation by three mechanisms: (i) regulation of the activity of factor I, which permits inactivation of protein C3b (iC3b); (ii) inhibition of formation of the alternative C3 convertase by competition with factor B for binding to C3b; (iii) acceleration of the dissociation of C3 convertase of the alternative pathway (C3bBb) (decay acceleration activity).

Factor H can act either in the fluid phase of the blood in order to maintain a low level of activated C3 molecules (C3b), or at cell surfaces presenting polyanions, such as glycosaminoglycans, heparan sulphate or sialic acid, in order to protect the host cells against cell lysis induced by the activation of complement.

The HF1 gene having 102,494 base pairs (bp) and 23 exons (NCBI RefSeq: NG_{—}007259.1), is localized in the RCA (regulator of complement activation) gene cluster on locus 1q32, Chr1. Through alternative splicing, two mRNAs are transcribed starting from the HF1 gene, one having 3696 bp and coding for the FH protein and the other having 1347 bp and coding for the FHL-1 protein (FH-like 1 protein).

The factor H protein (FH) (1213 aa, 155 kDa) is a single-chain glycoprotein, constituted by 20 repeated SCR (short consensus repeats) domains (also called CCP or SHUSHI). The FHL-1 protein (449 amino acids, 42 kDa) has the first 7 SCR domains plus 4 hydrophobic C-terminal amino acids.

Naturally, human factor H (FH) has a polymorphism in position 402 of its amino acid sequence, characterized by the substitution of a tyrosine (Y) with a histidine (H). This substitution itself results from a nucleotide replacement at the level of the HF1 gene, where a T nucleotide is replaced with a C.

The SCR domains are present in a great many proteins, such as proteins of the FH family (FHR1, FHR2, FHR3, FHR4), proteins of the RCA family (CR1, CR2, C4BP, CD55, CD46) or proteins of pathogens (vaccinia virus, etc.).

An SCR domain is composed of about 60 amino acids separated by a short sequence linker (3-8 amino acids for human FH). The SCR domains consist mainly of beta strands and they each possess two S—S bridges distributed at each end of the domain. Analyses of sequence homology show that 4 conserved cysteines are involved in the formation of two intradomain S—S bridges and that a tryptophan (W) in the SCR domain is highly conserved.

Owing to the great pharmacological therapeutic interest, many studies have been carried out in various systems for in vitro production of protein with the aim of improving the productivity of recombinant human factor H. Recombinant human factor H has been produced in the baculovirus system (Sharma and Pangburn, Gene 1994, Jun. 10: 143 (2): 301-2) and in COS cells (Sanchez-Corral et al., Am. J. Hum. Genet., 71: 1285-1295, 2002). More recently, recombinant human factor H was produced in the moss Physcomitrella in a photobioreactor (Buttner-Mainik et al., Plant Biotechnol. J. 2010 Aug. 17) as well as in the yeast Pichia pastoris (Schmidt C Q et al., Protein Expr. Purif. 2011 April 76(2) 254-63).

Despite these many efforts, to date, the productivity achieved for recombinant human factor H has not always been satisfactory for therapeutic use in humans. Consequently, there is still a great need to develop a high-performance method for producing human factor H.

One of the aims of the present invention is to propose the use of a human cell line for the production of recombinant human factor H.

One of the other aims of the present invention is to provide a method for preparing recombinant human factor H.

The present invention relates to the use of a human cell line for implementing a method for preparing recombinant human factor H with a yield greater than the quantity of endogenous factor H produced by said cell line.

In an embodiment, the present invention relates to the use of a human cell line for implementing a method for preparing recombinant human factor H, represented by the sequence SEQ ID NO: 1, or a sequence having at least 99%, in particular 99.4%, in particular 99.7% of sequence identity with sequence SEQ ID NO: 1.

A protein represented by such a sequence can be the Y402 variant or the H402 variant of human factor H or another variant of human factor H, such as a variant mentioned on the website http://www.uniprot.org/uniprot/P08603.

In an embodiment, the present invention relates to the use of a human cell line for implementing a method for preparing recombinant human factor H, represented by the sequence SEQ ID NO: 1, with a yield greater than the quantity of endogenous factor H produced by said cell line.

The recombinant human factor H represented by the sequence SEQ ID NO: 1 corresponds to the Y402 variant, in which the amino acid in position 402 is a tyrosine.

In another embodiment, the present invention relates to the use of a human cell line for implementing a method for preparing recombinant human factor H, represented by the sequence SEQ ID NO: 13, with a yield greater than the quantity of endogenous factor H produced by said cell line.

The recombinant human factor H represented by the sequence SEQ ID NO: 13 corresponds to the H402 variant, in which the amino acid in position 402 is a histidine.

This cell line can be a line that does not produce factor H, a line producing factor H in an quantity that is not detectable, or a line producing endogenous factor H.

The quantity of endogenous factor H produced by a cell line is determined by the ELISA method with a carefully selected pair of antibodies that makes it possible to detect minimum concentrations of 5 ng/ml of Factor H in the cellular supernatant. Commercial assay kits are also available (e.g. Kit HK342 Hycult).

More particularly, the present invention relates to the use of a human cell line producing endogenous factor H, for implementing a method for preparing recombinant human factor H, represented by the sequence SEQ ID NO: 1, with a yield greater than the quantity of endogenous factor H produced by said cell line.

By “a line producing factor H” is meant a cell line for which it is possible to detect the presence of endogenous factor H in the cellular supernatant by the standard methods for detection of proteins such as ELISA and Western Blot.

Certain human cell lines, such as the PER.C6® cell line or the HEK 293F line, produce human factor H by an endogenous route. However, the productivity of endogenous factor H by these cells is relatively low; it is about 70 μg/L for the PER.C6® cell line, after culture for 48 h, and about 11 μg/L of factor H secreted in the culture medium for the HEK 293F cell line after 7 days of culture.

In an advantageous embodiment, the present invention relates to the use of a human cell line, with a yield greater than 10 mg/L, in particular greater than 20 mg/L, or greater than 30 mg/L, or greater than 40 mg/L, or greater than 50 mg/L, more particularly greater than 60 mg/L, or greater than 70 mg/L, or greater than 80 mg/L, or greater than 90 mg/L, particularly greater than 100 mg/L of culture medium.

When the production mode used for producing recombinant factor H is one of the following modes: “batch”, “fed-batch” or “culture with filtration-retention” or a mode that is derived from one of these 3 modes of production, by “yield” is meant the quantity of recombinant factor H produced per volume of final culture medium that will be treated in post-production (also called Downstream Process or DSP).

When the production mode used for producing recombinant Factor H is a “continuous perfusion culture” mode or one that is derived from this production mode, by “yield” is meant the quantity of factor H produced per volume of culture medium contained initially in the production vessel.

The “batch” production mode is characterized by a constant volume. The inoculum is added to the medium and the culture is left to develop. The biomass develops according to the growth curve characteristic of the line used for production. Changes are made to the rotary speed, the aeration (O₂ and CO₂) or the adjustment of pH, but no nutrient medium is added during culture and nothing is withdrawn.

By way of illustration, a final quantity of recombinant proteins of 10 mg produced in a culture medium with a final volume of one litre, which is identical to the initial volume, corresponds to a production yield of 10 mg/L.

The “fed-batch” production mode is characterized by a variable volume. Production is carried out firstly in the form of a normal batch in a given volume. Fresh medium is then added in order to keep the cells in the desired state (stable cellular viability, exponential phase, stationary phase). It is possible to adapt the “feed” so as never to exceed the desired limits (example: glucose concentration <4 g/L).

The “feed” strategy depends on the line used for production. In the exponential phase, biomass accumulates rapidly, also allowing product to accumulate very rapidly in a reduced volume. In the stationary phase the biomass is kept constant, permitting gradual accumulation of product in a reduced volume.

By way of illustration, a final quantity of 30 mg of recombinant factor H produced in 1.5 litre of final volume of culture medium, which is initially one litre, corresponds to a production yield of 20 mg/L.

The production mode “culture with filtration-retention” (also known by the term “XD process”) is characterized by an increase of biomass in a constant volume. It is a mixed mode in the batch and fed-batch modes for which addition of fresh medium is compensated by the withdrawal of an equivalent volume of supernatant without recombinant Factor H, which will be retained by means of a filtration membrane whose cut-off is less than the size of the recombinant protein. The supernatant withdrawn is discarded. The volume thus remains constant whereas the biomass accumulates rapidly, also allowing recombinant Factor H to accumulate.

By way of illustration, a final quantity of 60 mg of recombinant factor H produced in one litre of final volume of culture medium, which is identical to the initial volume of culture medium, corresponds to a production yield of 60 mg/L.

The “culture with perfusion” production mode is characterized by a constant volume and a constant biomass. Just as for the filtration-retention mode, addition of fresh medium is compensated by the withdrawal of an equivalent volume of supernatant but in this production mode the supernatant removed contains recombinant Factor H and is therefore kept, which leads to a final volume to be treated in DSP greater than the initial volume without accumulation of the protein of interest.

As an example, a final quantity of 90 mg of recombinant factor H produced in 10 litres of final volume of culture medium, which is initially one litre, corresponds to a production yield of 90 mg/L.

Advantageously, the nucleic acids encoding respectively for the factor H represented by the sequence SEQ ID NO: 1 or SEQ ID NO: 13 have been subjected to the optimization of codons.

The present invention is based on the inventors' unexpected finding that the optimization of human factor H codons makes it possible to increase the productivity of recombinant human factor H in several human cell lines.

The aim of codon optimization is to replace the natural codons with codons whose transfer RNAs (tRNAs) bearing the amino acids are the most frequent in the cellular type considered. Mobilizing tRNAs that are frequently encountered has the major advantage of increasing the rate of translation of the messenger RNAs (mRNAs) and therefore of increasing the final titre (Carton J M et al., Protein Expr Purif, 2007). Sequence optimization also affects the prediction of the secondary structures of mRNA which might slow down reading by the ribosomal complex. Sequence optimization also has an impact on the percentage of G/C, which is directly linked to the half-life of the mRNAs and therefore to their potential for being translated (Chechetkin, J. of Theoretical Biology 242, 2006, 922-934).

Codon optimization can be carried out by substitution of the natural codons, using codon usage tables for mammals and more particularly for Homo sapiens. There are algorithms on the Internet, which are made available by the suppliers of synthetic genes (DNA2.0, GeneArt, MWG, Genscript), and which make it possible to carry out this sequence optimization.

In a more advantageous embodiment, the nucleic acid encoding for the sequence SEQ ID NO: 1 is represented by:

(i) sequence ID NO: 2,

(ii) a sequence having at least 75%, preferably at least 85%, in particular 90%, particularly 95% of sequence identity with sequence SEQ ID NO: 2.

The percentage identity between two nucleic acid sequences can be calculated from the following formula:

$$\frac{the number of identical residues \times 100}{the number of residues in the shorter sequence}$$

The sequence SEQ ID NO: 2 or a sequence having at least 75%, preferably at least 85%, in particular 90%, particularly 95% of sequence identity with sequence SEQ ID NO: 2 is a sequence obtained by codon optimization based on the natural nucleic acid sequence encoding for human factor H.

The natural nucleic acid encoding for the Y402 variant of human factor H is represented by the sequence SEQ ID NO: 8.

The natural nucleic acid encoding for the H402 variant of human factor H is represented by the sequence SEQ ID NO: 17.

In another more advantageous embodiment, the nucleic acid encoding for the precursor of human factor H further comprising a nucleic acid is selected from:
a nucleic acid represented by the sequence SEQ ID NO: 5 and encoding for the natural signal peptide of factor H, or
a nucleic acid represented by the sequence SEQ ID NO: 3 or by a sequence having at least 85%, in particular 90%, particularly 95% of sequence identity with sequence SEQ ID NO: 3, and encoding for the signal peptide of factor H, (optimized natural PS) or
a nucleic acid encoding for a natural signal peptide of a protein different from factor H, or
a nucleic acid encoding for the signal peptide encoded by the sequence SEQ ID NO: 4 (PCT/FR2011/050544) or by a sequence having at least 85%, in particular 90%, particularly 95% of sequence identity with sequence SEQ ID NO: 4.

The natural signal peptide of a protein different from human factor H can be a signal peptide selected from the signal peptides of all the proteins that are secreted in eukaryotes and in particular in mammals and more particularly in humans such as those of the immunoglobulins, of growth factors such as EPO, of hormones such as insulin, of enzymes such as trypsinogen, of clotting factors such as prothrombin.

The sequence SEQ ID NO: 3 or a sequence having at least 85%, in particular 90%, particularly 95% of sequence identity with sequence SEQ ID NO: 3 is a sequence obtained by codon optimization based on the sequence SEQ ID NO: 5.

The nucleic acid represented by the sequence SEQ ID NO: 4 or by a sequence having at least 85%, in particular 90%, particularly 95% of sequence identity with sequence SEQ ID NO: 4 encodes for an artificial signal peptide.

The presence of a signal peptide makes it possible to confer better secretion of recombinant protein in the culture medium.

In an embodiment, the whole of the nucleic acid encoding for the precursor of human factor H, comprising the nucleic acid encoding for the signal peptide and the nucleic acid encoding for human factor H, is subjected the optimization of codons.

In a particular embodiment, the nucleic acid encoding for the precursor of recombinant human factor H comprises or is constituted by:
a nucleic acid encoding for a signal peptide, said nucleic acid being selected from the sequence SEQ ID NO: 3 or a sequence having at least 85%, in particular 90%, particularly 95% of sequence identity with sequence SEQ ID NO: 3, and
a nucleic acid encoding for factor H represented by the sequence SEQ ID NO: 1, said nucleic acid being selected from the sequence SEQ ID NO: 2 or a sequence having at least 75%, preferably 85%, in particular 90%, particularly 95% of sequence identity with sequence SEQ ID NO: 2,
   said precursor permitting the expression of factor H represented by the sequence SEQ ID NO: 1.

More particularly, the nucleic acid encoding for the precursor of recombinant human factor H comprises or consists of the nucleic acid represented by the sequence SEQ ID NO: 3 and the nucleic acid represented by the sequence SEQ ID NO: 2.

Even more particularly, the nucleic acid encoding for the precursor of factor H is represented by the sequence SEQ ID NO: 6 or a sequence having at least 85%, in particular 90%, particularly 95% of sequence identity with sequence SEQ ID NO: 6.

In a particularly advantageous embodiment, the nucleic acid encoding for the precursor of factor H is represented by the sequence SEQ ID NO: 6.

The sequence SEQ ID NO: 6 corresponds the optimization of codons of the precursor of human factor H that comprises its natural signal peptide.

In another embodiment, the nucleic acid encoding for the precursor of recombinant human factor H comprises or is constituted by:
a nucleic acid encoding for a signal peptide, said nucleic acid being selected from the sequence SEQ ID NO: 3 or a sequence having at least 85%, in particular 90%, particularly 95% of sequence identity with sequence SEQ ID NO: 3, and
a nucleic acid encoding for factor H represented by the sequence SEQ ID NO: 13,
   said nucleic acid being selected from the sequence SEQ ID NO: 14 or a sequence having at least 75%, preferably 85%, in particular 90%, particularly 95% of sequence identity with sequence SEQ ID NO: 14,
   said precursor permitting the expression of factor H represented by the sequence SEQ ID NO: 13.

More particularly, the nucleic acid encoding for the precursor of recombinant human factor H comprises or consists of the nucleic acid represented by the sequence SEQ ID NO: 3 and the nucleic acid represented by the sequence SEQ ID NO: 14.

Even more particularly, the nucleic acid encoding for the precursor of factor H is represented by the sequence SEQ ID NO: 15 or a sequence having at least 85%, in particular 90%, particularly 95% of sequence identity with sequence SEQ ID NO: 15.

In another embodiment, a nucleic acid encoding for a precursor of recombinant human factor H comprises or is constituted by:
a nucleic acid that encodes for human factor H and has been subjected to the optimization of codons, and
a nucleic acid encoding for an artificial signal peptide, which does not correspond to the natural signal peptide of human factor H, but can confer a secretion capacity that is better than or is similar to that of the natural signal peptide of human factor H.

In another particular embodiment, the nucleic acid encoding for the precursor of recombinant human factor H comprises or is constituted by:
a nucleic acid encoding for a signal peptide, said nucleic acid being selected from the sequence SEQ ID NO: 4 or a sequence having at least 85%, in particular 90%, particularly 95% of sequence identity with sequence SEQ ID NO: 4, and
a nucleic acid encoding for the sequence SEQ ID NO: 1, said nucleic acid being selected from the sequence SEQ ID NO: 2 or a sequence having at least 75%, preferably at least 85%, in particular 90%, particularly 95% of sequence identity with sequence SEQ ID NO: 2,
   said precursor permitting the expression of factor H represented by the sequence SEQ ID NO: 1.

More particularly, the nucleic acid encoding for the precursor of recombinant human factor H comprises or is constituted by the nucleic acid represented by the sequence SEQ ID NO: 4 and the nucleic acid represented by the sequence SEQ ID NO: 2.

Even more particularly, the nucleic acid encoding for the precursor of recombinant human factor H is represented by the sequence SEQ ID NO: 7 or a sequence having at least 85%, in particular 90%, particularly 95% of sequence identity with sequence SEQ ID NO: 7, said precursor permitting the expression of factor H represented by the sequence SEQ ID NO: 1.

In a particularly advantageous embodiment, the nucleic acid encoding for the precursor of factor H is represented by the sequence SEQ ID NO: 7.

In another embodiment, the nucleic acid encoding for the precursor of recombinant human factor H comprises or is constituted by:
a nucleic acid encoding for a signal peptide, said nucleic acid being selected from the sequence SEQ ID NO: 4 or a sequence having at least 85%, in particular 90%, particularly 95% of sequence identity with sequence SEQ ID NO: 4, and
a nucleic acid encoding for factor H represented by the sequence SEQ ID NO: 13,
   said nucleic acid being selected from the sequence SEQ ID NO: 14 or a sequence having at least 75%, preferably 85%, in particular 90%, particularly 95% of sequence identity with sequence SEQ ID NO: 14,
   said precursor permitting the expression of factor H represented by the sequence SEQ ID NO: 13.

More particularly, the nucleic acid encoding for the precursor of recombinant human factor H comprises or is constituted by the nucleic acid represented by the sequence SEQ ID NO: 4 and the nucleic acid represented by the sequence SEQ ID NO: 14.

Even more particularly, the nucleic acid encoding for the precursor of factor H is represented by the sequence SEQ ID NO: 24 or a sequence having at least 85%, in particular 90%, particularly 95% of sequence identity with sequence SEQ ID NO: 24.

A nucleic acid encoding for the precursor of human factor H comprising or constituted by the nucleic acid represented by the sequence SEQ ID NO: 5 and the nucleic acid represented by the sequence SEQ ID NO: 8 can be used as a control of the productivity of an optimized nucleic acid encoding for the Y402 variant as described in the present invention.

More particularly, this control nucleic acid can be the nucleic acid represented by the sequence SEQ ID NO: 9.

A nucleic acid encoding for the precursor of human factor H comprising or constituted by the nucleic acid represented by the sequence SEQ ID NO: 5 and the nucleic acid represented by the sequence SEQ ID NO: 17 can be used as a control for an optimized nucleic acid encoding for the H402 variant as described in the present invention.

More particularly, this control nucleic acid can be the nucleic acid represented by the sequence SEQ ID NO: 18.

In an advantageous embodiment of the present invention, the human factor H as defined above is produced in the PER.C6® cell line or the HEK 293F cell line.

The PER.C6® cell line is derived from human primary retinal cells in which an adenoviral DNA fragment Ad5, containing both the E1A gene and the E1B gene, is inserted into the cells by a vector. This adenoviral DNA fragment makes it possible to confer immortality on the cells in which it is inserted, by virtue of the protein E 1B, which inhibits p53. As for the protein E1A, it has tropism for the viral promoter hCMV and permits its transactivation and potentiation of the genetic sequence that will be inserted at the 3′ end of the latter and which can be Factor H.

The PER.C6® cell line produces, endogenously, both the H402 variant of human factor H and the Y402 variant.

The present invention relates in particular to the use of the PER.C6® cell line for implementing a method for preparing recombinant human factor H, represented by the sequence SEQ ID NO: 1, with a yield greater than the quantity of endogenous factor H produced by said cell line.

More particularly, the present invention relates to the use of the PER.C6® cell line for implementing a method for preparing recombinant human factor H, represented by the sequence SEQ ID NO: 1, in which the nucleic acid encoding for the precursor of factor H is represented by the sequence SEQ ID NO: 6, 7 or 9.

The present invention also relates in particular to the use of the HEK 293F cell line for implementing a method for preparing recombinant human factor H, represented by the sequence SEQ ID NO: 1, with a yield greater than the quantity of endogenous factor H produced by said cell line.

The HEK 293F cell line produces, by an endogenous route, only the Y402 variant of human factor H.

More particularly, the present invention relates to the use of the HEK 293F cell line for implementing a method for preparing recombinant human factor H, represented by the sequence SEQ ID NO: 1, in which the nucleic acid encoding for the precursor of factor H is represented by the sequence SEQ ID NO: 6 or 7.

The present invention also relates to the use of a human cell line, such as the PER.C6® or HEK 293F cell line, for implementing a method for preparing recombinant human factor H, in which the nucleic acid encoding for the precursor of factor H is a genomic nucleic acid comprising artificial or natural introns of human factor H or of another human gene and natural exons of human factor H.

The present invention also relates to the nucleic acid encoding for a recombinant human factor H, such as the Y402 variant of factor H represented by the sequence SEQ ID NO: 1 or the H402 variant of factor H represented by the sequence SEQ ID NO: 13.

SEQ ID NO: 1 and SEQ ID NO: 13 do not contain the sequence corresponding to that of the signal peptide of factor H.

In an embodiment, the invention relates to a nucleic acid encoding for recombinant human factor H having the sequence SEQ ID NO: 1, comprising a nucleic acid represented by:

(i) the sequence SEQ ID NO: 2, or

(ii) a sequence having at least 75%, preferably at least 85%, in particular 90%, particularly 95% of sequence identity with sequence SEQ ID NO: 2.

In a particular embodiment, the invention relates to a nucleic acid encoding for the recombinant human factor H of sequence SEQ ID NO: 1, comprising or constituted by:
a nucleic acid encoding for a signal peptide, said nucleic acid being selected from the sequence SEQ ID NO: 3 or a sequence having at least 85%, in particular 90%, particularly 95% of sequence identity with sequence SEQ ID NO: 3, and
a nucleic acid encoding for the factor represented by the sequence SEQ ID NO: 1, said nucleic acid being selected from the sequence SEQ ID NO: 2 or a sequence having at least 75%, preferably 85%, in particular 90%, particularly 95% of sequence identity with sequence SEQ ID NO: 2.

More particularly, said nucleic acid encoding for the recombinant human factor H of sequence SEQ ID NO: 1 is represented by the sequence SEQ ID NO: 6 or a sequence having at least 85%, in particular 90%, particularly 95% of sequence identity with sequence SEQ ID NO: 6.

In another embodiment, the invention relates to a nucleic acid encoding for the recombinant human factor H of sequence SEQ ID NO: 1, comprising or constituted by:
a nucleic acid encoding for a signal peptide, said nucleic acid being selected from the sequence SEQ ID NO: 4 or a sequence having at least 85%, in particular 90%, particularly 95% of sequence identity with sequence SEQ ID NO: 4, and
a nucleic acid encoding for the factor represented by the sequence SEQ ID NO: 1, said nucleic acid being selected from the sequence SEQ ID NO: 2 or a sequence having at least 75%, preferably 85%, in particular 90%, particularly 95% of sequence identity with sequence SEQ ID NO: 2.

More particularly, said nucleic acid encoding for the recombinant human factor H of sequence SEQ ID NO: 1 is represented by the sequence SEQ ID NO: 7 or a sequence having at least 85%, in particular 90%, particularly 95% of sequence identity with sequence SEQ ID NO: 7.

In another embodiment, the invention relates to a nucleic acid encoding for the recombinant human factor H of sequence SEQ ID NO: 13, comprising or constituted by:
a nucleic acid encoding for a signal peptide, said nucleic acid being selected from the sequence SEQ ID NO: 3 or a sequence having at least 85%, in particular 90%, particularly 95% of sequence identity with sequence SEQ ID NO: 3, and
a nucleic acid encoding for the factor represented by the sequence SEQ ID NO: 1, said nucleic acid being selected from the sequence SEQ ID NO: 14 or a sequence having at least 75%, preferably 85%, in particular 90%, particularly 95% of sequence identity with sequence SEQ ID NO: 14.

More particularly, said nucleic acid encoding for the recombinant human factor H of sequence SEQ ID NO: 13 is represented by the sequence SEQ ID NO: 15 or a sequence having at least 85%, in particular 90%, particularly 95% of sequence identity with sequence SEQ ID NO: 15.

In another embodiment, the invention relates to a nucleic acid encoding for the recombinant human factor H of sequence SEQ ID NO: 13, comprising or constituted by:
a nucleic acid encoding for a signal peptide, said nucleic acid being selected from the sequence SEQ ID NO: 4 or a sequence having at least 85%, in particular 90%, particularly 95% of sequence identity with sequence SEQ ID NO: 4, and
a nucleic acid encoding for the factor represented by the sequence SEQ ID NO: 1, said nucleic acid being selected from the sequence SEQ ID NO: 14 or a sequence having at least 75%, preferably 85%, in particular 90%, particularly 95% of sequence identity with sequence SEQ ID NO: 14.

More particularly, said nucleic acid encoding for the recombinant human factor H of sequence SEQ ID NO: 1 is represented by the sequence SEQ ID NO: 24 or a sequence having at least 85%, in particular 90%, particularly 95% of sequence identity with sequence SEQ ID NO: 24.

In the present invention, SEQ ID NO: 16 is constituted by SEQ ID NO: 4 and SEQ ID NO: 19.

In the present invention, SEQ ID NO: 21 is constituted by SEQ ID NO: 4 and SEQ ID NO: 20.

In the present invention, SEQ ID NO: 22 is constituted by SEQ ID NO: 3 and SEQ ID NO: 20.

In the present invention, SEQ ID NO: 23 is constituted by SEQ ID NO: 3 and SEQ ID NO: 19.

In the present invention, SEQ ID NO: 19 corresponds to the optimized FH 402H sequence 2.

In the present invention, SEQ ID NO: 20 corresponds to the optimized FH 402Y sequence 2.

The present invention also relates to an expression vector comprising or constituted by a nucleic acid as defined above.

The present invention also relates to human cells, in particular transformed, producing recombinant human factor H with a yield greater than the quantity of endogenous factor H produced by said cells.

In a preferred embodiment, the invention relates to human cells having a yield greater than 10 mg/L, in particular greater than 30 mg/L, more particularly greater than 50 mg/L, particularly greater than 100 mg/L of culture medium.

In a particular embodiment, the invention relates to human cells transformed by a vector as defined above.

In a more particular embodiment, the invention relates to human cells producing recombinant factor H represented by the sequence SEQ ID NO: 1 with a yield greater than the quantity of endogenous factor H produced by said cells.

In an even more particular embodiment, human cells of the invention are selected from PER.C6® and HEK 293F.

In a particular embodiment, the human cells of the invention form a cellular clone.

The present invention also relates to a method for preparing recombinant human factor H, represented by the sequence SEQ ID NO: 1, in a human cell line with a yield greater than the quantity of endogenous factor H produced by said cell line, said method comprising the step of culture of said transformed cell line by a vector comprising a nucleic acid encoding for the precursor of human factor H.

The vector comprising such a nucleic acid can be any expression vector for eukaryotic cell lines known to a person skilled in the art.

The transformation of the cell line can be carried out by techniques of electroporation, of the AMAXA nucleofection type, using a “gene gun” or else using a transfection agent known to a person skilled in the art, such as cationic agents of liposomes or of polymers such as Fectin or PEI agent.

In a particular embodiment, the method according to the present invention comprises the following steps:

(i) transforming a cell line by means of an expression vector comprising a nucleic acid encoding for the precursor of human factor H, in order to obtain a transformed cell line,

(ii) culturing said transformed cell line, in order to obtain the expression of factor H in the culture medium.

Said expression vector can contain an antibiotic resistance gene in order to permit selection of the transfected cells during establishment of cells stably producing the protein of interest.

In a more particular embodiment, the method according to the present invention comprises the following steps:

(i) transforming a cell line, by means of a vector comprising a nucleic acid encoding for the precursor of the human factor H protein, in order to obtain a transformed cell line,

(ii) culturing said transformed cell line in order to obtain the expression of factor H in the culture medium,

(iii) purifying the human factor H from the culture medium, and

(iv) optionally separating the endogenous form and the recombinant form of the factor H purified in step (iii).

The human factor H is purified by chromatographic techniques in one, two or more steps. Purification in one step can be an ion exchange column or an affinity column (heparin, factor H ligand or anti-factor H antibody). Purification in two steps can be a step of chromatography on a cation exchange column followed by a step of chromatography on an anion exchange column or a step of chromatography on an anion exchange column followed by a step of chromatography on a cation exchange column or a step of chromatography on an ion exchange column followed by a step of chromatography on an affinity column or a step of chromatography on an affinity column followed by a step of chromatography on an ion exchange column. Purification in more than two steps can be carried out by a combination of these different types of chromatography. A step of diafiltration, ultrafiltration or gel filtration can be carried out additionally. The purity of a product after this purification can reach 99% of purified product.

More particularly, the vector for transforming the cell line comprises a nucleic acid encoding for the precursor of human factor H, represented by the sequence SEQ ID NO: 1, said nucleic acid comprising:

(i) a nucleic acid encoding for a signal peptide, said nucleic acid being selected from the sequence SEQ ID NO: 3 and a sequence having at least 85%, in particular 90%, particularly 95% of sequence identity with sequence SEQ ID NO: 3, and
a nucleic acid encoding for the sequence SEQ ID NO: 1, said nucleic acid being selected from the sequence SEQ ID NO: 2 and a sequence having at least 75%, preferably at least 85%, in particular 90%, particularly 95% of sequence identity with sequence SEQ ID NO: 2,

or

(ii)—a nucleic acid encoding for a signal peptide, said nucleic acid being selected from the sequence SEQ ID NO: 4 and a sequence having at least 85%, in particular 90%, particularly 95% of sequence identity with sequence SEQ ID NO: 4, and
a nucleic acid encoding for the sequence SEQ ID NO: 1, said nucleic acid being selected from the sequence SEQ ID NO: 2 and a sequence having at least 75%, preferably at least 85%, in particular 90%, particularly 95% of sequence identity with sequence SEQ ID NO: 2.

In an advantageous embodiment, the vector for transforming the cell line comprises a nucleic acid, which comprises:

(i) the nucleic acid represented by the sequence SEQ ID NO: 3 and the nucleic acid represented by the sequence SEQ ID NO: 2, or

(ii) the nucleic acid represented by the sequence SEQ ID NO: 4 and the nucleic acid represented by the sequence SEQ ID NO: 2.

In a more advantageous embodiment, the vector for transforming the cell line comprises a nucleic acid, which comprises:

(i) the nucleic acid encoding for the precursor of recombinant human factor H represented by the sequence SEQ ID NO: 6, or

(ii) the nucleic acid encoding for the precursor of recombinant human factor H represented by the sequence SEQ ID NO: 7.

The cell line used in the method according to the present invention can be the PER.C6® or the HEK 293F cell line.

A further objective of the present invention is to supply a recombinant factor H possessing a purity greater than 90%, preferably greater than 95%.

In a particular embodiment, said recombinant human factor H conserves the biological activity of a human plasma factor H.

The biological activity of human plasma factor H comprises the regulation of the activity of factor I, the inhibition of the formation of alternative C3 convertase and the acceleration of the dissociation of C3 convertase.

The methods for determining the above biological activities are known to a person skilled in the art.

In a more particular embodiment, said recombinant human factor H conserves the biological activity of a human plasma factor H for accelerating the dissociation of C3 convertase.

The quantity of C3 convertase dissociated can be determined as a function of the concentration of FH added. IC50 is determined from the equation calculated for a sigmoid of variable slope.

In another more particular embodiment, said recombinant human factor H conserves the biological activity of a plasma factor H for regulating the activity of factor I.

The present invention also relates to a pharmaceutical composition comprising solely a variant of recombinant human factor H as active ingredient, said variant being a variant mentioned on the website http://www.uniprot.org/uniprot/P08603.

In particular, the present invention also relates to a pharmaceutical composition comprising solely the Y402 variant of recombinant human factor H represented by the sequence SEQ ID NO: 1, without the H402 variant of human factor H.

The Y402 variant of recombinant human factor H can be produced by the HEK 293F cell line, which produces solely the Y402 variant endogenously.

The production of the recombinant Y402 variant can also be implemented by means of the PER.C6® cell line, which produces both the Y402 variant and the H402 variant endogenously, followed by purification or separation which makes it possible to separate the Y402 variant from the H402 variant. The PER.C6® cell line can also be modified so as not to produce the H402 variant endogenously.

The present invention also relates to a pharmaceutical composition comprising only the H402 variant of human factor H, without the Y402 variant represented by the sequence SEQ ID NO: 1.

The H402 variant of recombinant human factor H can be produced by the PER.C6® cell line or the HEK 293F cell line. Purification or separation is necessary in order to separate the H402 variant from the Y402 variant. The PER.C6® and HEK 293F cell lines can also be modified so as not to produce the Y402 variant endogenously.

The present invention also relates to a pharmaceutical composition comprising the H402 variant and the Y402 variant as active ingredient.

A pharmaceutical composition according to the present invention also comprises a pharmaceutically acceptable vehicle.

The present invention also relates to a pharmaceutical composition comprising more than 99.5% of one variant of human factor H and less than 0.5% of another variant of human factor H.

The present invention is shown in the figures and the examples given below. These figures and examples are not in any way intended to limit the scope of the present invention.

### FIGURES

FIG. 1 represents assay of endogenous factor H in the PER.C6® and HEK 293F cells.

FIG. 2 represents generation of stable PER.C6® pools expressing the recombinant factor H in a stable fashion (in this case, an example for the H402 variant of factor H).

FIG. 3 represents the kinetics of the production of recombinant factor H(H402) in the stable PER.C6® pools in batch mode.

FIG. 4 represents ELISA assay of the recombinant Factor H produced in the HEK 293F cells in batch mode for 7 days.

FIG. 5 represents ELISA assay of the recombinant Factor H produced in the stable PER.C6® pools in batch mode for 7 days.

FIG. 6A represents SDS-PAGE analysis of the recombinant Factor H produced in the supernatant of the PER.C6® and HEK 293F cells.

FIG. 6B represents Western Blot analysis of the recombinant Factor H produced in the supernatant of the PER.C6® and HEK 293F cells.

FIG. 6C illustrates the comparison of the electrophoretic profile of the purified recombinant human factor H produced in the PERC6 and HEK cells with that of the purified human plasma factor (batch LFB) in SDS-PAGE and staining with Coomassie Blue before and after treatment with a reducing agent (DTT).

FIG. 6D illustrates analysis of the purified recombinant human factor H produced in the PERC6 and HEK cells and purified human plasma factor H (batch LFB) by Western Blot using an anti-factor H polyclonal antibody (The Binding Site, ref: PC030).

FIG. 7A represents heparin column purification of factor H followed by a step of diafiltration.

FIG. 7B illustrates SDS-PAGE analysis and staining with Coomassie Blue of the purification steps in a step of cation exchange chromatography (SP-Sepharose) of the recombinant human factor H produced by the PERC6 cells. The starting supernatant (track 2), the different fractions obtained during the purification procedure (tracks 3-7, track 3: acidified supernatant, track 4: fraction not retained (FNA), track 5: washing, track 6: eluate SP, track 7: eluate NaCl 2M) and the final product (recombinant human FH Y402) purified, concentrated and filtered (track 8: main band at 120.6 kDa, 98.4%)) were deposited. The final product was also deposited under reducing conditions (track 9 main band at 143.5 kDa, 93.9%)). Tracks 1 and 10 are mass controls.

FIG. 7C illustrates SDS-PAGE analysis and staining with Coomassie Blue of the steps of purification in two steps of ion exchange chromatography (SP-Sepharose then Q-Sepharose) of the recombinant human factor H produced by the HEK cells. The starting supernatant (track 2), the different fractions obtained during the purification procedure (tracks 3-7 and 10-14: track 3: acidified supernatant, track 4: fraction not retained (FNA) 1st chromatography, track 5: washing 1st chromatography, track 6: eluate 1st chromatography, track 7: eluate 2M NaCl 1st chromatography, track 10: eluate 1st chromatography, track 11: fraction not retained (FNA) 2nd chromatography, track 12: washing 2nd chromatography, track 13: eluate 2nd chromatography, track 14: eluate 2M NaCl 2nd chromatography) and the final product (recombinant human FH Y402) purified, concentrated and filtered (track 15) were deposited. The final product was also deposited under reducing conditions (track 16). Tracks 1, 8, 9 and 17 are mass controls.

FIG. 8A represents determination of the activity of acceleration of the dissociation of C3 convertase in the presence of supernatant (Sn) of the PER.C6® and HEK 293F cells containing recombinant human factor H (Y402 variant or H402 variant).

FIG. 8B describes determination of the activity of acceleration of the dissociation of C3 convertase of the purified recombinant human Factor H produced in the PER.C6® and HEK 293F cells and human plasma Factor H.

FIGS. 9A and 9B illustrate analysis of the purified recombinant human factor H produced in the PERC6 and HEK cells by molecular sieving. 95 μg of the purified recombinant human factor H produced in PERC6 (FIG. 9A) and 50 μg of the purified recombinant human factor H produced in HEK (FIG. 9B) were injected separately on a gel filtration column (GE Healthcare, Superdex 200 10/300 GL, ref.: 17-5175-01). A main peak (99% and 97% respectively for PERC6 and HEK) with a size of 377-378 kDa corresponding to human factor H is observed.

FIGS. 10A and 10B illustrate capillary electrophoresis electrofocusing of the purified recombinant human factor H produced in the PERC6 (FIG. 10A) and HEK (FIG. 10B) cells.

FIGS. 11A and 11B illustrate analysis of the purified recombinant human factor H produced in the PERC6 and HEK cells by SDS gel capillary electrophoresis under non-reducing conditions (FIG. 11A) or under reducing conditions (FIG. 11B).

FIGS. 12A, 12B, 12C and 12D illustrate analysis of the cofactor activity of Factor I (FI) of the human recombinant FHs PERC6 and HEK and of the formulated human plasma factor H (form. FH) or in PBS 1× buffers (FH dess). 10 μg of protein C3b and 140 ng of factor I are incubated at 37° C. for 30 min in the presence of increasing quantities of factor H (50, 100, 250, 500 and 1000 ng). The various samples are deposited in SDS-PAGE in order to quantify the bands corresponding to the α′ and β chains of the C3b molecule and to the cleaved fragments α′1 and α′2 of the α′ chain. The percentage of C3b molecules inactivated is then calculated from the ratio of the total quantity of α′ chains cleaved to the total quantity of α′ chain. FIG. 13 shows the dose-response curve of inhibition of the lysis of sheep red blood cells by human plasma factor H (PB03 or LP03) or recombinant factor H (PERC6 or HEK).

### EXAMPLES

### Example 1

### Materials and Methods

1.1 Optimization of the Nucleic Acid Encoding for Human Factor H Sequence optimization was carried out with the algorithm from the supplier of synthetic genes with optimization for Homo sapiens avoiding the restriction sites required for molecular cloning.

1.2 Transfection of the pcDNA2001Neo Vectors Containing the Sequences of Factor H into the PER.C6® Cells for Generating Stable Pools

Transfection in stable pools is carried out in parallel with the empty (=without factor H sequence) pcDNA2001neo vector. The stable pool transfections are carried out according to the novel protocol described below.

Electroporation will be carried out with the following vectors:

| | Expression vector |
|---|---|
| control | pcDNA2001Neo |
| Factor H | MD1Y, MD1H |
| | MD2Y, MD2H |
| | MD3Y, MD3H |

MD1Y denotes the vector comprising the nucleic acid represented by the sequence SEQ ID NO: 9.
MD2Y denotes the vector comprising the nucleic acid represented by the sequence SEQ ID NO: 6.
MD3Y denotes the vector comprising the nucleic acid represented by the sequence SEQ ID NO: 7.
MD1H corresponds to the vector comprising the nucleic acid represented by the sequence SEQ ID NO: 18.
MD2H corresponds to the vector comprising the nucleic acid represented by the sequence SEQ ID NO: 15
MD3H corresponds to the vector comprising the nucleic acid represented by the sequence SEQ ID NO: 24.

Pre-Culture of the Cells

The cells for stable pool transfection, originating from suspension culture starting from cells in suspension stored in cryotubes in serum-free medium, are adapted PER.C6 SF cells in Permab medium (Hyclone, ThermoFisher Scientific) with stirring. They were cultured for 3 weeks with stirring at 125 rpm.

Two days before electroporation, the cells were put in suspension at 5^{E}5 vc/mL by complete replacement of the Permab medium. The volume is adjusted to the number of cuvettes planned during electroporation.

On the day of electroporation, the cell concentration and the viability are determined. The cells must be in the exponential growth phase and must have a viability greater than 90%.

Before electroporation, the Permab medium (3 mM L-glutamine) is preheated to ambient temperature.

Electroporation

The following steps are carried out in 6 cuvettes:

Pre-aliquot 60 mL in a T150 and preheat to 37° C. (i.e. 10 mL per cuvette)

The Following Steps are Carried Out in Each Cuvette:
Centrifuge 6^{E}6 vc at 300 g for 5 minutes and discard the supernatant
Resuspend the cells, shaking the tube about ten times
Add 400 μL of Permab medium (3 mM L-glutamine) at ambient temperature, without leaving the cells for more than 15 minutes.
Add 8 μg of DNA into a sterile 1.5 mL Eppendorf tube
Carefully add 400 μL of cellular suspension to the 8 μg of DNA
Transfer the DNA/Cells suspension to a 4 mm electroporation cuvette. Check that there are no bubbles at the bottom of the cuvette.
After transferring the cuvette to the electroporation chamber, apply the following BioRad Gene Pulser Xcell programme:

| | |
|---|---|
| Voltage (V) | 250 |
| Pulse duration (ms) | 5 |
| Number of pulses | 1 |
| Pulse interval (s) | 0 |
| Cuvette (mm) | 4 |

Remove the white aggregates floating on the surface (cellular debris) and carefully remove the cellular suspension using a 1 mL pipette. Add the cells directly to the 60 mL of medium preheated to 37° C. Do not do this dropwise, to avoid the formation of precipitates.
Begin the following steps again until the 6 cuvettes are obtained for generating the PER.C6® stable pool.

Selection and Generation of a Stable Pool

48 hours after transfection, the efficacy of transfection is evaluated. The cell culture is resuspended and a count is carried out on 1 mL. The concentration of viable cells divided by the starting concentration (6E5 vc/mL) gives the percentage coverage of the viability.

A container with 5E5 vc/mL is seeded by complete replacement of the medium and the addition of Permab (3 mM L-glutamine) and G418 at 125 μg/mL. All of the cells are centrifuged at 300 g for 5 min before the addition of the selective medium.

The cells are passaged twice weekly (on Monday and Friday) by the complete renewal of the selective medium. The volume and the container are adapted so as to obtain a concentration of 3E5 vc/mL at each passage. After 2-3 weeks, cellular viability improves. When the latter reaches about 50%, cultures under stirred conditions can be initiated. In this case, the starting concentration is 5E5 vc/mL. When the culture reaches more than 85% viability, cryopreservation is carried out at 5E6 vc/ampoule.

Seeding of the Batch Culture for Production of Recombinant Factor H:

The volume of the container used for the batch is 250 ml with an initial working volume of 30 ml. Any change in volume of the container leads automatically to an adaptation of the volumes and values stated in this protocol.

The cells are seeded at a concentration of 1^{E}6 vc/ml by complete renewal of the culture medium. The cells are incubated for 7 days at a temperature of 36.5° C., stirring at 125 rpm without renewal or addition of medium. A sample is taken every day between the 3rd and 7th day of culture for determining the viability, the cell density and the production of recombinant factor H.

On the 7th day, the cells are centrifuged at 3000 g for 15 minutes. The cellular deposit is removed and the cellular supernatant containing the recombinant factor H is filtered at 0.22 μm and then frozen at −20° C.

1.3: Transient Transfection into HEK 293F Cells for the Transient Production of Recombinant Factor H

Pre-Culture of the Cells:

On the day before transient transfection, the HEK 293F cells are subcultured at a cellular concentration of 7^{E}5 vc/ml.

Transient Transfection:

The cell density and the viability of the HEK 293F cells are determined on the day of transfection. The culture volume corresponding to 30^{E}6 vc/ml is centrifuged. The supernatant is removed and the deposit of cells is taken up in 28 ml of F17 culture medium (Invitrogen), transferred to a 250 ml conical flask and incubated at 37° C.

Formation of the transfection agent/DNA complex in 2:1 ratio: The transfection agent (Fectin or PEI) and the DNA corresponding to the vector pCEP4 containing one of the factor H sequences are prepared in OptiMEM medium (Invitrogen) as follows:
Addition of 30 μg of DNA to 1 ml of OptiMEM
Addition of 60 μl of Fectin or 60 μl of PEI to 1 ml of OptiMEM.

These two preparations are incubated separately for 5 minutes at ambient temperature and then the solution containing the transfection agent is added carefully to the solution containing the DNA. The mixture is incubated for 25 minutes at ambient temperature before being added to the 28 ml of HEK 293F cells prepared previously.

The cells are then incubated at 37° C., with stirring at 125 rpm.

Positive transfection control: a vector expressing GFP (Green Fluorescent Protein) is also transfected under the same conditions. 24 h post-transfection the efficacy of transfection is determined by fluorescence microscopy (ratio of the number of cells expressing GFP to the total number of cells).

Growth control: HEK 293F cells are transfected under the same conditions but without an expression vector. This control makes it possible to determine the post-transfection viability and whether there is toxicity associated with the transient production of the exogenous protein (recombinant factor H).

Transient Production of Factor H:

The cells are maintained in culture for 7 days without the addition or renewal of culture medium. The cell density and the viability are determined every day between the 5th and the 7th day.

On the 7th day, the cells are centrifuged at 3000 g for 15 minutes. The cellular deposit is removed and the cellular supernatant containing the recombinant factor H is filtered at 0.22 μm and then frozen at −20° C.

1.4 Test of Cell Viability

The cell density and the viability are determined using an automatic cell culture analyser (Cedex, Innovatis—Roche Applied Science). Measurement of the viability is based on counting the cells that have incorporated trypan blue.

1.5 Characterization of the Recombinant Human Factor H

SDS-PAGE:

Preparation of samples: the equivalent of 1 μg of protein is mixed v/v with Laemli 2× buffer. The mixture is heated for 5 minutes at 95° C. in order to denature the proteins.

The proteins are then deposited in the wells of a linear gel 10% Bis-Tris/HCl pH 6.4. Migration is carried out in the presence of a MOPS buffer (50 mM MOPS, 50 mM Tris Base, 0.1% SDS, 1 mM EDTA, pH 7.7). The proteins are separated at a constant voltage of 200 volts for 60 minutes.

Staining with Coomassie Blue:

After migration, the gel is washed 3×5 min with distilled water and then stained with a solution of Coomassie Blue overnight. The excess is removed by successive washings with distilled water. A photograph of the gel is then taken using an imager.

Detection by Western Blot:

After migration, the gel is transferred to a buffer containing 40 mM of amino-6-hexanoic acid, Tris 25 mM pH 9.8.

On a semi-dry transfer apparatus (TE77, Amersham Pharmacia), a sandwich is made from the anode to the cathode as follows:
6 Whatman papers preincubated in Tris 0.3M buffer pH 10.4
3 Whatman papers preincubated in Tris 25 mM buffer pH 10.4
1 nitrocellulose membrane (RPN 303E, GE Healthcare) preincubated in Tris 25 mM buffer pH 10.4
the gel preincubated in the buffer 40 mM of amino-6-hexanoic acid, Tris 25 mM pH 9.8
9 Whatman papers preincubated in the buffer 40 mM of amino-6-hexanoic acid, Tris 25 mM pH 9.8

Transfer is carried out at constant current density of 0.8 mA/cm² of membrane for 60 minutes.

After transfer, the membrane is saturated overnight at 4° C. in a PBS buffer containing 1% of BSA and 0.1% of Tween 20. The membrane is then washed with physiological saline containing 0.1% of Tween 20. The factor H is detected using an anti-factor H monoclonal antibody (MCA 508G, Serotec) at a concentration of 0.5 μg/ml incubated at ambient temperature for 60 minutes, followed by a peroxidase-coupled anti-mouse IgG secondary antibody at a concentration of 80 ng/ml incubated at ambient temperature for 60 minutes. The labelled proteins are then revealed using a chemiluminescence enzymatic kit and detected using an imager equipped with a photon detector.

1.6 Assay of Human Factor H in the Culture Medium by ELISA

Buffer and Solutions:

1—Coating Buffer: PBS
Dissolve the contents of one sachet of salts for PBS buffer, pH 7.4 (Sigma: P-3813 or salts of equivalent quality) in one litre of WFI.

2—Coating Antibody: Sheep Anti-Human Factor H Immunoglobulins (the Binding Site—Ref.: PC030)
Dilute the antibody extemporaneously in the coating buffer to obtain a concentration between 3.5 and 5.5 μg/ml.

3—Saturation Buffer: PBS Buffer—BSA 1% (w/v)
For one plate, dissolve 0.15 g of BSA (BSA: Sigma—Ref.: A7030 or Jackson Immuno Research Laboratories, Ref.: 001-000-162 or a BSA of equivalent quality) in 15 ml of PBS buffer.

4—Washing Buffer: PBS Buffer—Tween 20 0.1% (v/v)
Add 1 ml of Tween 20 to 1 litre of PBS.

5—Dilution Buffer: PBS Buffer—Tween 20 0.1% (v/v)—BSA 0.1% (w/v)
Buffer for dilution of the standard, of the samples for assay and of the detection antibody. Dissolve the saturation buffer at 1/10th in washing buffer.

6—Standard Solution: Human Factor H Calibrator NL (the Binding Site, Ref: RP030)
Take up the lyophilizate in the volume of WFI indicated by the supplier and wait 30 min, then distribute into 20 μl aliquots and freeze them at −70° C. Dilute the standard to 1/7000th (for a std at 700 mg/L), in order to obtain a stock solution of 100 ng/ml, i.e.:
1st pre-dilution to 1/70th: 5 μl of Std+345 μl of dilution buffer,
2nd pre-dilution to 1/100th: 5 μl of the 1st pre-dilution+495 μl of dilution buffer.
Then dilute half-and-half in the dilution buffer at 7 points.
The blank is performed with the dilution buffer.

7—Samples
The samples are pre-diluted so as to obtain a concentration close to that of the first point of the range. Then dilute half-and-half at 8 points.

8—Anti-Factor H Monoclonal Antibody: Purified Anti-Human Factor H Mouse Monoclonal Immunoglobulins (SEROTEC—Ref.: MCA508G)
Dilute the antibody extemporaneously in the dilution buffer to 1/5000th, i.e. for 1 plate: 2 μl in 10 ml of dilution buffer.

9—Detection Antibody: Peroxidase-Labelled Anti-Mouse IgG Goat Immunoglobulins (Jackson Immuno Research Laboratories, Ref.: 115-035-062).
Dilute the antibodies extemporaneously in the dilution buffer to 1/10000th ( 1/50th then 1/200th) i.e. for one plate: 1/50th: 2 μl in 98 μl of dilution buffer, then 1/200th: 50 μl in 10 ml of dilution buffer.

10—Detection Solution: TMB Kit
Mix the solution of peroxidase substrate (3,3′,5,5′-tetramethylbenzidine) and the solution of peroxide extemporaneously, volume by volume.

11—Stopping Solution: 4 N or 2 M Sulphuric Acid (Fisher Scientific, Ref.: 0379D).

Procedure

Sensitization of the Solid Phase
In a microtitre plate, distribute 100 μl per well of diluted coating antibody.
Cover with an adhesive film.
Incubate overnight at +4° C. and empty the plate by inverting it.

Saturation of the Solid Phase
Distribute 120 μl per well of saturation solution.
Cover with an adhesive film.
Incubate for 1 h at 20° C.
Wash 3 times in washing buffer.

Antigen Capture
Distribute 100 μl per well of dilution buffer (blank), of each dilution of the standard and of the samples.
Cover with an adhesive film.
Incubate for 1.5 h at 20° C.
Wash 5 times in washing buffer.

Recognition of the Antigen by the Monoclonal Antibody
Distribute 100 μl per well of the monoclonal antibody.
Cover with an adhesive film.
Incubate for 1.5 h at 20° C.
Wash 5 times in washing buffer.

Labelling with HRP (Horseradish Peroxidase) Conjugate
Distribute 100 μl per well of the detection antibody.
Cover with an adhesive film.
Incubate for 1 hour at 20° C.
Wash 5 times in washing buffer.

Enzymatic Reaction
Distribute 100 μl per well of TMB containing the substrate at regular time intervals and away from any intense light.
Incubate at ambient temperature for 5-15 min.
This time must be identical for all the assay points.

Stopping the Reaction
Distribute 100 μl of 4NH₂SO₄ per well at regular time intervals.
Read the optical densities (OD) at 450 nm.

Utilizing the Results

The linear regression straight line of the standard curve is defined by the pairs (Log (OD at 450 nm-OD blank), Log of the concentration of factor H in ng/ml).

The result is the mean value of all the results obtained for one and the same sample in the linear portion of the straight line obtained for the range of the standard.

1.7 Characterization of the Purified Recombinant Human Factor H

The recombinant human factor H produced in the PER.C6® line is purified by ion exchange chromatography in one step.

The recombinant human factor H produced in the HEK 293 line is purified by ion exchange chromatography in two steps.

The results of the SDS-PAGE analysis of the steps of purification of the recombinant factor H produced by the PER.C6® or HEK 293 cells are illustrated in FIGS. 7B and 7C.

The presence of the purified recombinant human factor H is revealed by SDS-PAGE gel that is stained with Coomassie Blue and by Western blot according to section 1.5 described above.

The results of analysis of the recombinant human FH purified by SDS-PAGE or by Western Blot are shown in FIGS. 6C and 6D respectively.

The recombinant human factor H thus purified is analysed by molecular sieving, by capillary electrophoresis electrofocusing or by SDS gel capillary electrophoresis according to the methods described below.

Molecular Sieving

1. Procedure

50 to 100 μg of recombinant human factor H is injected on a column of Superdex 200 10/300 GL filtration gel (GE Healthcare, ref.: 17-5175-01) equilibrated beforehand in PBS1X buffer. Separation is carried out at a constant flow rate of 0.4 ml/min on FPLC apparatus coupled to a UV detector (GE Healthcare, Akta Prime).

The result of the analysis by molecular sieving is shown in FIGS. 9A and 9B.

2. Results

The chromatogram obtained with the purified recombinant FH produced by the HEK 293F cell line has a major peak having a retention time of 27.88 min, which corresponds to the FH (FIG. 9A)

The chromatogram obtained with the purified recombinant FH produced by the PER.C6 cell line has a major peak having a retention time of 27.89 min which corresponds to the FH and a minor peak which emerges at 21.07 min corresponding to aggregated forms of the recombinant FH (FIG. 9B).

Capillary Electrophoresis Electrofocusing

1. Procedure

The isoforms of the recombinant FHs are determined by capillary electrophoresis using the Advanced cIEF Starter kit (Beckman Coulter, A80976) and the PA800 capillary electrophoresis apparatus with a UV detector (Beckman Coulter).

75 μg of each sample of recombinant FH to be analysed is desalted by centrifugation on Zeba Desalt Spin columns (Pierce), dried in the Speedvac at ambient temperature and then taken up in a volume of ultra-pure water so as to obtain a concentration of protein from 5 to 10 mg/ml.

10 μl of each sample is then mixed with 240 μL of mix constituted by 200 μl of 3M urea, 12 μl of Pharmalyte 3-10 carrier ampholytes, 20 μl of cathodic stabilizer, 2 μl of anodic stabilizer and 2 μl of pI markers between 4.1 and 10.

The sample preparations are injected on an eCAP™ capillary (Beckman Coulter, 477441) using the pre-programmed method Basic pH Gradient Separation Method met. In order to verify the linearity of migration as a function of the pI values, a mixture of 5 markers (from pI 4.1 to 10) is injected at the beginning and at the end of the analysis sequence.

2 Analysis of the Results and Calculation of the pI Values

After integration of the peaks of the electropherogram, the software 32 Karat automatically calculates the pI values of the sample. The result is shown in FIGS. 10A and 10B.

The analysis shows the presence of several isoforms the experimental pI of which varies slightly (between 5.53 and 6.01 and between 5.86 and 6.13 for PERC6 and HEK respectively) with values close to the theoretical pI of human FH, which is 6.12 (see the table below).

Human FH produced in the PER.C6 line

| | pI | | | | | | | | | |
|---|---|---|---|---|---|---|---|---|---|---|
| | 6.01 | 5.97 | 5.89 | 5.85 | 5.83 | 5.75 | 5.64 | 5.61 | 5.59 | 5.53 |
| Area in % | 0.9 | 1.8 | 0.2 | 2.6 | 1.9 | 3.5 | 21.3 | 9.5 | 23.0 | 35.4 |

Human FH produced in the HEK line

| | pI | | | | | | | | |
|---|---|---|---|---|---|---|---|---|---|
| | 6.13 | 6.1 | 6.09 | 6.05 | 6.02 | 5.95 | 5.92 | 5.89 | 5.86 |
| Area in % | 15.5 | 8.7 | 2.6 | 0.7 | 14.6 | 10.5 | 27.0 | 3.2 | 17.2 |

### SDS Gel Capillary Electrophoresis:

1. Procedure:
The molecular weights of the recombinant FHs are determined by capillary electrophoresis using the SDS-MW Analysis kit (Beckman Coulter, 390953) and the PA800 capillary electrophoresis apparatus with a UV detector (Beckman Coulter). 200 μg of each sample of recombinant FH to be analysed is mixed with 150 μl of sample buffer (100 mM Tris-HCl pH 9.0, 1% SDS) and centrifuged on Centricon YM-10 (Millipore, PN 4205) at 4000 g for 10 min. The operation is repeated 2 times (addition of 150 μl of sample buffer and centrifugation at 4000 g for 5 min) and then the volume is made up to 100 μl with sample buffer.
For the analysis under non-reduced conditions, 50 μl of the sample is taken and mixed with 45 μl of sample buffer, 2 μl of internal standard 10 kD, 5 μl of 250 mM iodo-acetamide and is heated at 70° C. for 10 min. For analysis under reduced conditions, the 50 μl that remains is mixed with 45 μl of sample buffer, 2 μl of internal standard 10 kD, 5 μl of 2-mercaptoethanol and is heated at 70° C. for 10 min.
A solution of standard molecular weight is also prepared by mixing 85 μl of sample buffer, 2 μl of SDS-MW size marker (10 to 225 kD) and 5 μl of 2-mercaptoethanol.
The samples thus prepared are analysed by injection on a 50 μm I.D. capillary bare-fused silica 2 using the pre-programmed method SDS MW Conditioning-PA800 plus-met. On the basis of the standard calibration curve, the molecular weight of the samples is determined by the software 32 Karat. Integration of the peaks makes it possible to calculate the percentage that each peak of the sample represents.

2. Analysis of the Results:

Based on the calibration curve of the standard, the molecular weight of the samples is determined using the software 32 Karat. Integration of the peaks makes it possible to calculate the proportions of each peak of the sample. The result is shown in FIGS. 11A and 11B and the following table.

The electrophoretic profile shows the presence of a very major peak (>95%), which corresponds to the purified human factor H and whose size is 173-176 kDa under non-reducing conditions (FIG. 11A) and 143-148 kDa under reducing conditions (FIG. 11B).

| | | HEK | | | | PER.C6 | |
|---|---|---|---|---|---|---|---|
| Non- | pM | 16 | 29 | 66 | 173 | 48 | 176 |
| reducing | (kDa) | | | | | | |
| conditions | Area in % | 0.5 | 0.6 | 1.2 | 97.7 | 0.3 | 99.7 |
| Reducing | pM | 16 | 36 | 66 | 145 | 46 | 143 |
| conditions | (kDa) | | | | | | |
| | Area in % | 0.8 | 0.6 | 1.0 | 95.6 | 1.3 | 98.7 |

1.8 Characterization of the Biological Activity of Recombinant Human Factor H

1.8.1 Anti-C3 Convertase Activity (C3 Convertase Decay-Accelerating Activity)

Buffers and Solutions

Coating Buffer

0.1M carbonate buffer—pH 9.6

Na₂CO₃: 5.3 g in 500 ml of WFI (water for injection) 0.1M

NaHCO₃: 4.2 g in 500 ml of WFI 0.1M

Add 30 ml of the Na₂CO₃ solution to 70 ml of the NaHCO₃ solution and adjust the pH to 9.6 if necessary. Store at 4° C. for 2 months.

Washing Buffers

a) PBS buffer pH 7.4 (Sigma)+Tween 20 0.1% (=Buffer A)

Dissolve 0.1 ml of Tween 20 in 100 ml of PBS buffer pH 7.4.

Store at +4° C. (maximum 2 days).

b) Sodium phosphate buffer 10 mM, NaCl 25 mM, pH 7.2±0.05 (=Buffer B)

Dissolve in one litre of WFI: 1.56 g of NaH₂PO₄.2H₂O.

Dissolve in one litre of WFI: 3.58 g of Na₂HPO₄.12H₂O.

Add about 66 ml of the Na₂HPO₄ solution to 34 ml of the NaH₂PO₄ solution. Adjust the pH to 7.30. Add the NaCl required in order to obtain a final concentration at 25 mM. This buffer can be stored at 4° C. for 1 month.

This buffer serves as the base for the saturation, dilution and washing buffers.

To obtain the washing buffer (=Buffer C):

Dissolve 0.1 ml of Tween 20 in 100 ml of buffer B.

Store at +4° C. (maximum 2 days).

Saturation Buffer

Prepare a sufficient volume starting from buffer B. Add 1% of BSA (w/v).

Store for a maximum of 2 days at +4° C.

Dilution Buffer D

Prepare the necessary volume with buffer B, to which Tween 20 is added at a concentration of 0.05% and BSA at a concentration of 4% (w/v).

Store for a maximum of 2 days at +4° C.

Dilution Buffer E

Prepare dilution buffer E starting from PBS pH 7.4, to which 0.1% of BSA (w/v) is added.

Store for a maximum of 2 days at +4° C.

Solution of NiCl₂ 20 mM, NaCl 25 mM

Weigh 0.475 g of NiCl₂ q.s. 100 ml of a solution of NaCl 25 mM (store for a maximum of 3 months at +4° C.).

Substrate Buffer

Citrate buffer 0.1M+H₂O₂ 30%.

Citrate Buffer:

Dissolve 29 g of sodium citrate and 4.1 g of citric acid in one litre of WFI.

Adjust the pH to 5.5 if necessary.

Store at +4° C. for a maximum of 3 months.

Add 10 μl of H₂O₂ 30% to 10 ml of citrate buffer extemporaneously on the day it is used, and store protected from the light.

Stopping Solution

Solution of 4N (2M) sulphuric acid to be prepared or ready for use.

Prepare one litre: 100 ml H₂SO₄ 18M+900 ml of WFI.

Store at ambient temperature for a maximum of 6 months.

Procedure

Coating the Plates

Dilute C3b in the volume required for the test in the coating buffer to give a final concentration at 2.5 μg/ml (10 ml for one 96-well plate). Example: for batch D28449 at 1 mg/ml, dilute 25 μl in 10 ml of coating buffer.

Distribute 100 μl of the prepared solution into each well.

Cover the plate with an adhesive.

Incubate the plate for 1-1.25 h at +34° C. and then overnight at +4° C.

Washings

Wash the plate manually with a multidistributor micropipette (3×280 μl of washing buffer C). An automatic washer can also be used for this operation.

Empty by inverting.

Saturation

Distribute 300 μl of saturation buffer per well.

Incubate for 1-1.25 h at +34° C.

Empty by inverting.

Convertase Generating Step.

Prepare the following solution (10 ml for one 96-well plate):
Solution of NiCl₂ 20 mM, NaCl 25 mM: 750 μl (final concentration NiCl₂: 1.5 mM)
Factor B Calbiochem (1 mg/ml): 40 μl (final concentration: 4 μg/ml)
Factor D Calbiochem (0.1 mg/ml): 30 μl (final concentration: 0.3 μg/ml)
Dilution buffer D: 9180 μl.

Deposit 100 μl/well and incubate for 2 hours at +34° C.

Washings

Wash the plate manually with a multidistributor micropipette (3×280 μl of washing buffer C). An automatic washer can also be used for this operation.

Empty by inverting.

Preparation of the Ranges of Factor H

The ranges of factor H are prepared starting from a pool plasma (ECQ 1) and a reference batch or any sample containing factor H to be assayed. The Elisa antigen concentration or OD 280 serves as a basis for establishing the ranges by non-independent successive dilutions. The choice of assay method is as follows:
Elisa technique for the Pool Plasma and the samples of intermediate purity.
Measurement of OD 280 for the reference batch, a PV or a rehydrated final product.
Incubate for 32 to 34 minutes max. at +34° C.

Washings

Wash the plate manually with a multidistributor micropipette (3×280 μl of washing buffer 3.3.2 b). An automatic washer can also be used for this operation.

Empty by inverting.

Incubation of the Anti-Human Factor B Antibody

For 10 ml of dilution buffer E, add 5 μl of the anti-human factor B antibody (Calbiochem ref. 341272) and then distribute 100 μl/well (dilution to 1/2000).

Incubate for 1-1.25 h at 34° C.

Washings

Wash the plate manually with a multidistributor micropipette (3×280 μl of washing buffer A). An automatic washer can also be used for this operation.

Empty by inverting.

Incubation of the Peroxidase-Labelled Anti-Goat IgG (H+L) Antibody.

The dilutions are carried out with dilution buffer E:
pre-dilution to 1/40 (10 μl of antibody+390 μl of buffer)
dilution to 1/500 (20 μl of the pre-dilution+9980 μl of buffer).

Distribute 100 μl/well and incubate at ambient temperature for 25-30 min protected from the light.

Washings

Wash the plate manually with a multidistributor micropipette (3×280 μl of washing buffer A). An automatic washer can also be used for this operation.

Empty by inverting.

Enzymatic Reaction

Distribute 100 μl of TMB per well with a multichannel pipette, at regular intervals.

Incubate the plate for 5 to 15 minutes at ambient temperature, protected from the light.

Stopping the Reaction

Distribute 100 μl of the stopping solution into each well, at regular intervals.

Reading

Read the plate extemporaneously at 450 nm on a microplate reader after stirring gently.

Utilization of the Results

Elimination of Outliers

The absorbance values obtained in duplicate for each concentration of the range of factor H for the reference substance, the ECQ or for any other sample are averaged after eliminating the outliers. For example, for the values in duplicate of the samples, of the reference substance or of the ECQ, the elimination of one point per range and per concentration level is acceptable. Selection of the point or points to be eliminated makes it possible to improve the value of the correlation coefficient (R2) and the precision of the 95% confidence interval of the EC 50 obtained.

Using the Prism Software (Logi Labo)

This software makes it possible to determine the IC50 value of each sample in a non-linear modelled system (sigmoid with variable slope). The equation for calculating this model is as follows:

Y:Bottom+(Top−Bottom)/(1+10̂((Log IC50−X)*Hillslope)

X is the logarithm of the concentration (μg/ml).

Y is the response (OD).

Y starts at the baseline (Bottom) and goes to the top (Top) according to a sigmoidal shape. Hillslope is the slope.

The mean value of absorbance of each dilution obtained for the ECQ or sample (range in duplicate) is entered in the database for each corresponding concentration.

Analysis of the data is supplemented with the function “Runs test” for estimating the deviation of the test relative to the model selected.

The standard deviation of 1050 is given for a confidence interval of 95%.

Examination of the curves makes it possible to visualize all of the samples with one another and compare them with the ECQ.

Acceptance Criteria of the Assay

The AOD of the range of all the samples (including reference and ECQ) must be at least 0.35. Any lower value leads to automatic rejection of the test.

Acceptance of this condition then makes it possible to determine the respective value of their 1050.

The mean value of the 1050 of the pool plasma is 0.058 μg/ml.

The assay is valid with a tolerance of two standard deviations, which gives a mean value of 0.058±0.030 μg/ml. Every new ECQ must be tested in parallel with the previous one over at least ten assays in order to determine its characteristics.

The mean value of the 1050 of the reference substance and the confidence interval are determined after validation.

The value of the correlation coefficient must be at least 0.99 for each sample.

The result for the PER.C6® or HEK293F culture supernatant containing recombinant human FH is shown in FIG. 8A and the following table.

| | PURIFIED | | | | | | | | | | | |
|---|---|---|---|---|---|---|---|---|---|---|---|---|
| | PROTEIN | | PLASMA or CULTURE SUPERNATANT | | | | | | | | | |
| | Formulated | Plasma | FH plasma | PERC6 | PERC6 | PERC6 | | HEK | HEK | HEK | PERC6 TPS | HEK |
| | plasma | FH PBS | calibrator (The | MD2- | MD3- | MD2- | HEK MD2- | MD3- | MD1- | MD2- | empty | empty |
| | FH | 1X | binding site) | Y402 | Y402 | H402 | Y402 | Y402 | Y402 | H402 | vector | vector |
| IC50 (ng/ml) | 17.85 | 15.25 | 68.12 | 48.72 | 55.86 | 54.56 | 50.72 | 52.78 | 53.76 | 52.03 | ND | ND |

The result for the activity of the purified factor H is shown in FIG. 8B and the following table.

| | | | | |
|---|---|---|---|---|
| IC50 (ng/ml) | 54.57 | 43.84 | 45.11 | 50.64 |
| | Formulated | Purified | Purified | Purified |
| | purified | FH plasma | FH PER.C6 | FH HEK |
| | FH plasma | PBS 1X | | |

1.8.2 Cofactor Activity of Factor I:

Procedure

The cofactor activity of the FH is determined in a liquid-phase cleavage assay of C3b. C3b (10 μg) and FI (140 ng) are incubated alone or in the presence of FH (250 ng) in a volume of 100 μl in imidazole 20 mM, NaCl 75 mM buffer, pH 7.3 for 30 min at 37° C. The reaction is stopped by adding Laemmli sample buffer supplemented with DTT and then the various samples are subjected to polyacrylamide gel electrophoresis as described above. After staining with Coomassie Blue, the gels are scanned and analysed with the Quantity-one software in order to determine the relative intensities of each of the bands present in each track.

Summary Table of the Mixtures Performed

| | | Factor H | Factor I |
|---|---|---|---|
| | C3b | (50 μg/ml) | (11 μg/ml) |
| Control C3b | 10 μg | — | — |
| Control C3b + Factor H | 10 μg | 0.25 μg | — |
| Control C3b + Factor I | 10 μg | — | 0.14 μg |
| Control Factor H | — | 0.25 μg | — |
| Control Factor I | — | — | 0.14 μg |
| Samples | 10 μg | From 500 | 0.14 μg |
| | | to 15.6 ng | |

Results:

C3b has a molecular weight of 176 kDa and is composed of 2 chains joined together by a disulphide bridge: the α′ chain of 101 kDa and the β chain of 75 kDa. The α′ chain is cleaved by factor I into 2 fragments called α′1 (63 kDa) and α′2 (39 kDa); the β chain (75 kDa) remains unchanged.

In order to be able to compare the results obtained, the relative intensities are standardized relative to the intensity of the β band present in the track of the control C3b alone.

The percentage inactivation of the C3b is then calculated from the ratio of the cleaved α′ chains ((α′1+α′2)/2) to the total α′ chains: (α′+((α′1+α′2)/2)) (FIG. 12).

Analysis of the results presented in FIG. 12D shows that the recombinant FHs produced by the PER.C6 and HEK 293F lines have a similar cofactor activity of FI and that this activity is comparable to that of the human plasma FH.

### Example 2

### Construction of a Vector Comprising pcDNA2001Neo-MD1

The pcDNA2001neo-MD1 vector comprises the nucleic acid represented by the sequence SEQ ID NO: 8, which corresponds to the natural sequence of this human factor H.

The pUC57-MD1 vector sent by Genscript contains the synthetic gene corresponding to the nucleic acid represented by the sequence SEQ ID NO: 8 (MD1, natural sequence of human factor H). Digestion of the pUC57-MD1 vector by the NotI and BamHI restriction enzymes produces 2 fragments of 3000 bp and 3700 bp. These two fragments are separated by gel purification and Nucleospin extraction.

The pcDNA2001-neo vector is digested by the NotI and BamHI restriction enzymes, which produces 2 fragments of 25 bp and of 5533 bp. After extraction by Nucleospin and dephosphorylation, the 3700 bp fragment corresponding to the nucleic acid represented by the sequence SEQ ID NO: 8 (MD1) is inserted into the digested pcDNA2001-neo vector.

After ligation, the vector thus obtained is transformed into TOP10 bacteria.

Insertion of the DNA fragment corresponding to MD1 is verified by PCR screening, using the primers CMV1 (SEQ ID NO: 10 5′-CCATTGACGTCAATGGGAGTTTG-3′) and MD1-1rev (SEQ ID NO: 11 5′-GGTAAACACTTCACAACTTCACATATAG-3′), which gives a 758 bp band. The bacterial clones that are positive in PCR screening are then sequenced to verify unity of transcription of the factor H contained within the expression vector.

### Example 3

### Construction of the Vectors PcDNA2001Neo-MD2 and

pcDNA2001neo-MD3 comprising the nucleic acid sequences of the optimized factor H MD2 and MD3

The vectors pcDNA2001neo-MD2 and pcDNA2001neo-MD3 comprise the nucleic acid represented by the sequences SEQ ID NO: 2 and SEQ ID NO: 3 respectively, which correspond to the optimized sequence of human factor H and the optimized sequence of human factor H with the optimized signal peptide MB7.

The vectors pUC57-MD2 and pUC57-MD3 sent by Genscript contain the synthetic genes corresponding respectively to the nucleic acid represented by the sequences SEQ ID NO: 2 (MD2) and SEQ ID NO: 3 (MD3). They are digested by the NotI and BamHI restriction enzymes, which generates 2 nucleic acid fragments of 3000 bp and of 3700 bp. For each vector, these two fragments are separated by gel purification and Nucleospin extraction.

The pcDNA2001-neo vector is digested by the NotI and BamHI restriction enzymes, which generates 2 fragments of 25 and of 5533 bp. After Nucleospin extraction and dephosphorylation, the 3700 bp fragment corresponding to the coding sequence MD2 or MD3 is inserted into the digested pcDNA2001-neo vector.

After ligation, the vector thus obtained is transformed into TOP10 bacteria.

Insertion of MD2 or MD3 fragment is verified by PCR screening, using the primers CMV1 (SEQ ID NO: 10 5′-CCATTGACGTCAATGGGAGTTTG-3′)/MD2-1rev (SEQ ID NO: 12 5′-TGTCACACTCGCGGTAGTTG-3′), which gives a 706 bp band.

### Example 4

### Generation of stable PER.C6® pools transfected with pcDNA2001neo-MD1 or pcDNA2001neo-MD2 or pcDNA2001neo-MD3 (FIG. 2)

The PER.C6® cells are transfected with the pcDNA2001neo-MD1 vector or the pcDNA2001neo-MD2 or pcDNA2001neo-MD3 vector to generate stable pools according to the protocol described in the section given above (Example 1.2)

There is no significant difference in viability between the cells transformed by different vectors.

### Example 5

### ELISA Assay of the Recombinant Factor H (Y402 and H402 Variants) Produced in the Culture Supernatant of the PER.C6® and HEK 293F Cells (FIGS. 3, 4 and 5)

The culture supernatant is first diluted to 1/500th, the point starting from which a dilution range is prepared half-and-half at 8 points. Each point of the range and samples for assay are performed in duplicate. The assay is carried out according to the protocol described in the section given above (example 1.6).

### Example 6

### Characterization by SDS-PAGE and Western Blot, of the Recombinant Factor H Present in the Culture Supernatant or Purified (Y402 and H402 Variants) (FIGS. 6A, 6B and 7)

Starting from the concentration of recombinant factor H determined by ELISA or measurement of absorbance at 280 nm (purified protein), a volume corresponding to 1 μg of recombinant factor H is diluted to ½ in Laemmli 2× buffer, heated at 95° C. for 5 minutes and then deposited on a linear gel of 10% polyacrylamide. After migration, the gel is treated either for staining of the proteins with Coomassie Blue or for transfer to nitrocellulose membrane followed by immunolabelling (Western Blot) as described above (example 1.5).

### Example 7

### Determination of the Activity of Acceleration of the Dissociation of C3 Convertase of the Recombinant Factor H (Y402 and H402 Variants)

Based on the concentration of recombinant factor H determined by ELISA or measurement of absorbance at 280 nm (purified protein), the recombinant factor H is diluted to a final concentration of 20 μg/ml. Starting from this tube, a range is prepared by the following successive dilutions: 1/2; 1/10; 1/4; 1/4; 1/4; 1/4; 1/40. This range of decreasing concentration of factor H is added to the C3 convertase complex formed in the wells of a 96-well plate, which is then incubated for 32 to 34 minutes at +34° C. After washing several times, the factor B that is still complexed with the C3 molecule immobilized at the bottom of the well is then assayed by an immuno-enzyme reaction of the ELISA type. The absorbance values obtained as a function of the concentration of factor H added are then treated as indicated in the protocol described above (example 1.8). The activity determined in the cellular extract supernatant or after purification is shown in FIG. 8A (supernatant) and FIG. 8B (purified), respectively.

### Example 8

### Determination of the Cofactor Activity of Factor I of the Recombinant Human FHs

The cofactor activity of factor I (FI) of the recombinant human FHs produced in the PERC6 or HEK line and that of the formulated plasma FH or in PBS 1× buffers (desalted) are measured by the method described in section 1.8.2. 10 μg of C3b protein and 140 ng of factor I are incubated at 37° C. for 30 min in the presence of increasing quantities of factor H (50, 100, 250, 500 and 1000 ng). The various samples are deposited in SDS-PAGE in order to quantify the bands corresponding to the α′ and β chains of the C3b molecule as well as to the cleaved fragments α′1 and α′2 of the α′ chain. The percentage of C3b molecules inactivated is then calculated from the ratio of the quantity of α′ chains cleaved to the total quantity of α′ chain. The results are shown in FIGS. 12A, 12B, 12C and 12D.

### Example 9

### Test for Assaying the Anti-Haemolytic Activity of Human Factor H on Sheep Red Blood Cells (Sanchez-Corral Test)

This test makes it possible to measure the functional activity of the C-terminal portion of the recombinant human factor H purified during the development of therapeutic batches by evaluating its capacity for protecting sheep red blood cells from lysis induced by a serum depleted or deficient in functional factor H. This test is adapted from the “Sanchez-Corral” method for measuring the anti-haemolytic activity of the factor H present in the plasma of patients with haemolytic uremic syndrome. (P. Sanchez-Corral, C. González-Rubio, S. Rodriguez de Cordoba and M. Lopez-Trascasa. Molecular Immunology 41 (2004) 81-84).

In this connection, a mixture of serum depleted of factor H and of a human pool plasma is prepared in equal proportions in order to create the conditions of a specific lysis. The addition of purified recombinant human factor H provides protection of the sheep red blood cells (absence of cell lysis) against lysis induced by complement. Without CFH, spontaneous lysis of the erythrocytes is 30%.

The inhibition of lysis of the sheep erythrocytes by the rCFH is dose-dependent and reaches 100% inhibition at a dose of 4 μg. There is no difference between the two rCFHs but there is inhibitory activity well above that of the plasma CFH.

The following are added successively to 40 μl of a suspension of sheep erythrocytes (1×10⁸ erythrocytes/ml): 20 μl of the reaction buffer (Hepes 10 mM, NaCl 144 mM, MgCl_{2 7} mM, EGTA 10 mM, pH 7.2), 2 μl of FH or of PBS, then 9 μl of a pool of human plasma followed by 9 μl of human plasma depleted of FH. After incubation for 30 min at 37° C., 400 μl of cold HBS-EDTA buffer (Hepes 10 mM, NaCl 144 mM, EDTA 2 mM, pH 7.2) is added to stop the reaction. After centrifugation for 5 min at 1730 g, 200 μl of supernatant is taken, and is deposited in a microtitre plate to measure the absorbance at 414 nm.

The percentage lysis is determined from the formula:

$$\frac{{OD}_{414 nm} Reaction tube - {OD}_{414 nm} Blank tube}{{OD}_{414 nm} 100 \% lysis} \times 100$$

The “100% lysis” control corresponds to the maximum lysis of the sheep red blood cells observed in the presence of water.

The blank control comprises the reaction buffer+50 mM EDTA and corresponds to spontaneous lysis of the sheep red blood cells.

The result of this test is shown in the following table and in FIG. 13.

| | | | | |
|---|---|---|---|---|
| Factor H | | | | |
| added | LP03 | PB03 | PerC 6 | HEK |

| | |
|---|---|
| (μg) | Lysis (%) |

| | | | | |
|---|---|---|---|---|
| 0 | 33.75 | 33.75 | 33.75 | 33.75 |
| 2 | 32.6 | | 14.8 | 7.8 |
| 3 | 23.8 | | 2.4 | 1 |
| 4 | 11.1 | 10.2 | −1 | −0.8 |
| 6 | 0.9 | 1.1 | −0.2 | −0.6 |
| 8 | 0.3 | −1.5 | −0.9 | −1.4 |

1. A method for preparing recombinant human factor H with a yield greater than the quantity of endogenous factor H produced by a cell line, comprising:
culturing said cell line transformed by a vector comprising a nucleic acid encoding for the precursor of human factor H.

2. A human cell line producing endogenous factor H for implementing a method for preparing recombinant human factor H, represented by the sequence SEQ ID NO: 1, or a variant possessing a percentage homology of at least 99% with the sequence SEQ ID NO: 1, with a yield greater than the quantity of endogenous factor H produced by said cell line.

3. The method according to claim 1, with a yield greater than 10 mg/L, in particular greater than 30 mg/L, more particularly greater than 50 mg/L, particularly greater than 100 mg/L of culture medium.

4. The method according to claim 1, in which the recombinant human factor H, represented by the sequence SEQ ID NO: 1, and the codons of recombinant human factor H represented by the sequence SEQ ID NO: 1 are optimized relative to the natural codons of human factor H.

5. The method according to claim 4, in which the nucleic acid encoding for the sequence SEQ ID NO: 1 is represented by:
(i) the sequence SEQ ID NO: 2,
(ii) a sequence having at least 75%, preferably at least 85%, in particular 90%, particularly 95% of sequence identity with sequence SEQ ID NO: 2.

6. The method according to claim 5, in which the nucleic acid encoding for the precursor of factor H further comprises a nucleic acid selected from:
a nucleic acid represented by the sequence SEQ ID NO: 5 and encoding for the natural signal peptide of factor H,
a nucleic acid represented by the sequence SEQ ID NO: 3 or by a sequence having at least 85%, in particular 90%, particularly 95% of sequence identity with sequence SEQ ID NO: 3, and encoding for the signal peptide of factor H,
a nucleic acid encoding for a natural signal peptide of a protein different from factor H, or
a nucleic acid encoding for the signal peptide encoded by the sequence SEQ ID NO: 4 or by a sequence having at least 85%, in particular 90%, particularly 95% of sequence identity with sequence SEQ ID NO: 4.

7. The method according to claim 6, in which the nucleic acid encoding for the precursor of factor H comprises:
(i)—a nucleic acid encoding for a signal peptide, said nucleic acid being selected from the sequence SEQ ID NO: 3 and a sequence having at least 85%, in particular 90%, particularly 95% of sequence identity with sequence SEQ ID NO: 3, and
a nucleic acid encoding for the sequence SEQ ID NO: 1, said nucleic acid being selected from the sequence SEQ ID NO: 2 or a sequence having at least 75%, preferably at least 85%, in particular 90%, particularly 95% of sequence identity with sequence SEQ ID NO: 2,
or
(ii)—a nucleic acid encoding for a signal peptide, said nucleic acid being selected from the sequence SEQ ID NO: 4 or a sequence having at least 85%, in particular 90%, particularly 95% of sequence identity with sequence SEQ ID NO: 4, and
a nucleic acid encoding for the sequence SEQ ID NO: 1, said nucleic acid being selected from the sequence SEQ ID NO: 2 and a sequence having at least 75%, preferably at least 85%, in particular 90%, particularly 95% of sequence identity with sequence SEQ ID NO: 2,
said precursor permitting the expression of factor H represented by the sequence SEQ ID NO: 1.

8. The method according to claim 7, in which the nucleic acid encoding for the precursor of factor H comprises:
(i) the nucleic acid represented by the sequence SEQ ID NO: 3 and the nucleic acid represented by the sequence SEQ ID NO: 2, or
(ii) the nucleic acid represented by the sequence SEQ ID NO: 4 and the nucleic acid represented by the sequence SEQ ID NO: 2.

9. The method according to claim 7, in which the nucleic acid encoding for the precursor of factor H is represented by:
(i) the sequence SEQ ID NO: 6 or a sequence having at least 85%, in particular 90%, particularly 95% of sequence identity with sequence SEQ ID NO: 6, or
(ii) the sequence SEQ ID NO: 7 or a sequence having at least 85%, in particular 90%, particularly 95% of sequence identity with sequence SEQ ID NO: 7,
said precursor permitting the expression of factor H represented by the sequence SEQ ID NO: 1.

10. The method according to claim 9, in which the nucleic acid encoding for the factor H precursor is represented by the sequence SEQ ID NO: 6, or the sequence SEQ ID NO: 7.

11. Nucleic acid encoding for recombinant human factor H having the sequence SEQ ID NO: 1, comprising a nucleic acid represented by:
(i) the sequence SEQ ID NO: 2,
(ii) a sequence having at least 75%, preferably at least 85%, in particular 90%, particularly 95% of sequence identity with sequence SEQ ID NO: 2.

12. Nucleic acid encoding for the recombinant human factor H according to claim 11, comprising:
a nucleic acid encoding for a signal peptide, said nucleic acid being selected from the sequence SEQ ID NO: 3 and a sequence having at least 85%, in particular 90%, particularly 95% of sequence identity with sequence SEQ ID NO: 3, and
a nucleic acid encoding for the factor represented by the sequence SEQ ID NO: 1, said nucleic acid being selected from the sequence SEQ ID NO: 2 or a sequence having at least 75%, preferably 85%, in particular 90%, particularly 95% of sequence identity with sequence SEQ ID NO: 2.

13. Nucleic acid encoding for recombinant human factor H according to claim 12, represented by the sequence SEQ ID NO: 6 or a sequence having at least 85%, in particular 90%, particularly 95% of sequence identity with sequence SEQ ID NO: 6.

14. Nucleic acid encoding for recombinant human factor H according to claim 11, comprising:
a nucleic acid encoding for a signal peptide, said nucleic acid being selected from the sequence SEQ ID NO: 4 and a sequence having at least 85%, in particular 90%, particularly 95% of sequence identity with sequence SEQ ID NO: 4, and
a nucleic acid encoding for the factor represented by the sequence SEQ ID NO: 1, said nucleic acid being selected from the sequence SEQ ID NO: 2 or a sequence having at least 75%, preferably 85%, in particular 90%, particularly 95% of sequence identity with sequence SEQ ID NO: 2.

15. Nucleic acid encoding for the recombinant human factor H according to claim 14, represented by the sequence SEQ ID NO: 7 or a sequence having at least 85%, in particular 90%, particularly 95% of sequence identity with sequence SEQ ID NO: 7.

16. Expression vector comprising a nucleic acid according to claim 11.

17. Human cells, in particular transformed, producing recombinant human factor H with a yield greater than the quantity of endogenous factor H produced by said cells.

18. Human cells according to claim 17, having a yield greater than 10 mg/L, in particular greater than 30 mg/L, more particularly greater than 50 mg/L, particularly greater than 100 mg/L of culture medium.

19. Human cells, transformed by a vector according to claim 17.

20. Human cells according to claim 19, producing recombinant factor H represented by the sequence SEQ ID NO: 1 with a yield greater than the quantity of endogenous factor H produced by said cells.

21. Human cells according to claim 17, said cells being selected from PER.C6® and HEK 293F.

22. Human cells according to claim 17, said cells forming a cellular clone.

23. Method for preparing recombinant human factor H, represented by the sequence SEQ ID NO: 1, in a human cell line with a yield greater than the quantity of endogenous factor H produced by said cell line, said method comprising the step of:
(i) culturing said cell line transformed by a vector comprising a nucleic acid encoding for the precursor of human factor H.

24. Method according to claim 23, in which said method comprises the following steps:
(i) transforming a cell line with a vector comprising a nucleic acid encoding for the precursor of human factor H, to obtain a transformed cell line,
(ii) culturing said transformed cell line, in order to obtain the expression of factor H in the culture medium.

25. Method according to claim 24, in which said method comprises the following steps:
(i) transforming a cell line with a vector comprising a nucleic acid encoding for the precursor of the human factor H protein, in order to obtain a transformed cell line,
(ii) culturing said transformed cell line in order to obtain the expression of factor H in the culture medium,
(iii) purifying the human factor H from the culture medium, and
(iv) optionally separating the endogenous form and the recombinant form of the factor H purified in step (iii).

26. Method according to claim 23, in which the vector comprises a nucleic acid encoding for the precursor of human factor H, represented by the sequence SEQ ID NO: 1, said nucleic acid comprising:
(i)—a nucleic acid encoding for a signal peptide, said nucleic acid being selected from the sequence SEQ ID NO: 3 and a sequence having at least 85%, in particular 90%, particularly 95% of sequence identity with sequence SEQ ID NO: 3, and
a nucleic acid encoding for the sequence SEQ ID NO: 1, said nucleic acid being selected from the sequence SEQ ID NO: 2 and a sequence having at least 75%, preferably at least 85%, in particular 90%, particularly 95% of sequence identity with sequence SEQ ID NO: 2,
or
(ii)—a nucleic acid encoding for a signal peptide, said nucleic acid being selected from the sequence SEQ ID NO: 4 and a sequence having at least 85%, in particular 90%, particularly 95% of sequence identity with sequence SEQ ID NO: 4, and
a nucleic acid encoding for the sequence SEQ ID NO: 1, said nucleic acid being selected from the sequence SEQ ID NO: 2 and a sequence having at least 75%, preferably at least 85%, in particular 90%, particularly 95% of sequence identity with sequence SEQ ID NO: 2.

27. The method according to claim 23, in which said cell line is the PER.C6 cell line or the HEK 293F cell line.

28. Recombinant factor H, characterized in that it has a purity greater than 90%, preferably greater than 95%.

29. Recombinant factor H according to claim 28, characterized in that said recombinant factor H conserves the biological activity of a plasma factor H.

30. Recombinant factor H according to claim 29, characterized in that said recombinant factor H conserves the biological activity of a plasma factor H for accelerating the dissociation of C3 convertase.

31. Recombinant factor H according to claim 29, characterized in that said recombinant factor H conserves the biological activity of a plasma factor H for regulating factor I.

32. Pharmaceutical composition comprising as active ingredient a recombinant factor H as defined in claim 28 and a pharmaceutically acceptable vehicle.


SEQUENCE LISTING (24 sequences)

SEQ ID NO: 1 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - DNA, 3645 bp, Artificial Sequence
  Optimized FH
gaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 3 - DNA, 54 bp, Artificial Sequence
  Optimized FH PS
atgagactgctggctaagattatttgcctgatgctgtgggctatttgcgtcgcc

SEQ ID NO: 4 - DNA, 54 bp, Artificial Sequence
  PS MB7
atgcgatggtcttggatttttctgctgctgctgtctatcacttctgctaacgct

SEQ ID NO: 5 - DNA, 54 bp, Homo sapiens
atgagacttctagcaaagattatttgccttatgttatgggctatttgtgtagca

SEQ ID NO: 6 - DNA, 3699 bp, Artificial Sequence
  MD2
atgagactgctggctaagattatttgcctgatgctgtgggctatttgcgtcgccgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 7 - DNA, 3699 bp, Artificial Sequence
  MD3
atgcgatggtcttggatttttctgctgctgctgtctatcacttctgctaacgctgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 8 - DNA, 3639 bp, Homo sapiens
gaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggatccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggacccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaaga

SEQ ID NO: 9 - DNA, 3693 bp, Homo sapiens
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggatccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggacccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaaga

SEQ ID NO: 10 - DNA, 23 bp, Artificial Sequence
  CMV1 primer
ccattgacgtcaatgggagtttg

SEQ ID NO: 11 - DNA, 28 bp, Artificial Sequence
  MD1-1rev
ggtaaacacttcacaacttcacatatag

SEQ ID NO: 12 - DNA, 20 bp, Artificial Sequence
  MD2-1rev
tgtcacactcgcggtagttg

SEQ ID NO: 13 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 14 - DNA, 3645 bp, Artificial Sequence
  Optimized FH 402H
gaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 15 - DNA, 3699 bp, Artificial Sequence
  FH MD2 402H
atgagactgctggctaagattatttgcctgatgctgtgggctatttgcgtcgccgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 16 - DNA, 3699 bp, Artificial Sequence
  FH MD3 402H
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 17 - DNA, 3645 bp, Homo sapiens
gaagattgcaatgaacttcctccaagaagaaatacagaaattctgacaggttcctggtctgaccaaacatatccagaaggcacccaggctatctataaatgccgccctggatataggtcccttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggacccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatgataa

SEQ ID NO: 18 - DNA, 3699 bp, Homo sapiens
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatataggtcccttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggacccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatgataa

SEQ ID NO: 19 - DNA, 3645 bp, Artificial Sequence
  Optimized FH 402H, 2
gaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 20 - DNA, 3645 bp, Artificial Sequence
  Optimized FH 402Y, 2
gaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 21 - DNA, 3699 bp, Artificial Sequence
  FH MD3 402Y
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 22 - DNA, 3699 bp, Artificial Sequence
  FH 402Y (2)
atgagactgctggctaagattatttgcctgatgctgtgggctatttgcgtcgccgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 23 - DNA, 3699 bp, Artificial Sequence
  FH 402H (2)
atgagactgctggctaagattatttgcctgatgctgtgggctatttgcgtcgccgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagatgataa

SEQ ID NO: 24 - DNA, 3699 bp, Artificial Sequence
  FH 402 variant MD2
atgcgatggtcttggatttttctgctgctgctgtctatcacttctgctaacgctgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaatcacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa